(19) United States
(12) Patent Application Publication
(10) Pub. No.: US 2024/0261294 A1
(43) Pub. Date: Aug. 8, 2024

(54) METHOD FOR TREATING IDH INHIBITOR-RESISTANT SUBJECTS

(71) Applicant: Eli Lilly and Company, Indianapolis, IN (US)

(72) Inventors: Nathan Arthur BROOKS, Indianapolis, IN (US); Raymond GILMOUR, Indianapolis, IN (US)

(21) Appl. No.: 18/565,484

(22) PCT Filed: Jun. 9, 2022
(86) PCT No.: PCT/US2022/032774

§ 371 Date: Nov. 29, 2023

Related U.S. Application Data
(60) Provisional application No. 63/208,625, filed on Jun. 9, 2021.

(51) Int. Cl. A61K 31/5365; A61P 35/00

(52) U.S. Cl. CPC A61K 31/5365; A61P 35/00

(57) ABSTRACT

The present invention relates to the treatment of human cancer subjects with mutant isocitrate dehydrogenase inhibitors disclosed herein.

The present invention relates to the treatment of cancer in subjects with mutant isocitrate dehydrogenase (IDH) inhibitors disclosed herein.

IDH1 and IDH2 are enzymes that catalyze the conversion of isocitrate to α-ketoglutarate, and reduce nicotinamide adenine dinucleotide phosphate (NADP⁺) to NADPH (Megias-Vericat J, et al., Blood Lymph. Cancer: Targets and Therapy 2019; 9: 19-32).

Neomorphic (de novo) mutations in IDH1, e.g., at IDH1 amino acid residue R132, contribute to tumorigenesis in several types of cancer, including solid tumor cancers and hematologic malignancies (Badur M G, et al., Cell Reports 2018; 25: 1680). IDH1 mutations can result in high levels of 2-hydroxyglutarate (2-HG), which inhibits cellular differentiation, and inhibitors of mutant IDH1 can reduce 2-HG levels, which promotes cellular differentiation (Molenaar R J, et al., Oncogene 2018; 37: 1949-1960). Mutations also occur in IDH2, e.g., at amino acid residues R140 and R172 (Yang H, et al., Clin. Cancer. Res. 2012; 18: 5562-5571; Mondesir J, et al., J. Blood Med. 2016; 7: 171-180).

For example, acute myeloid leukemia (AML) is characterized by a diverse spectrum of mutated genes and a multi-clonal genomic architecture comprising preleukemic and leukemic clones that evolve dynamically over time and under the selective pressure of therapy (Bloomfield C D, et al., Blood Revs. 2018: 32: 416-425).

Induction chemotherapy with cytarabine and an anthracycline (“7+3”) has been the standard of care for more than 4 decades for subjects with newly diagnosed AML. In recent years, the following drugs have been approved by the U.S. Food and Drug Administration for treating AML: midostaurin, enasidenib, CPX-351, and gemtuzumab ozogamicin (Bloomfield C D, et al., Blood Revs. 2018; 32: 416-425), and ivosidenib (Megias-Vericat J, et al., Blood Lymph. Cancer: Targets and Therapy 2019; 9: 19-32).

Approximately 60% to 70% of adults with AML can be expected to attain complete remission (CR) status following appropriate induction therapy, and more than 25% of adults with AML (about 45% of those who attain CR) can be expected to survive 3 or more years and may be cured.

Mutations in the enasidenib binding site (IDH2-Q316E and IDH2-1319M) have been identified in IDH2 mutant AML patients relapsing on enasidenib (Intlekofer et al., “Acquired resistance to IDH inhibition through trans or cis dimer-interface mutations.” Nature 559, 125-129 (2018)). Preclinical studies demonstrated that enasidenib was no longer able to bind and inhibit the mutant enzymes (Intlekofer et al., 2018). These so called “secondary” IDH2 mutations, as defined herein, may contribute to relapse after treatment with a mutant IDH2 inhibitor.

Thus, there remains a need for alternative mutant IDH2-related cancer therapies, particularly for subjects in whom secondary IDH2 mutations occur after initial mutant IDH2 inhibitor therapy.

Certain mutant IDH1 and IDH2 inhibitors are disclosed in WO 2018/111707 A1, including a compound defined herein as “Compound A,” which is a covalent inhibitor of mutant IDH1 that modifies a single cysteine (Cys269) in an allosteric binding pocket, rapidly inactivates the enzyme, and selectively inhibits 2-HG production, without affecting (α-KG) levels (WO 2018/111707 A1).

The present invention provides a method for treating cancer, comprising administering to a human cancer subject having an IDH2 R140 or IDH2 R172 mutation, and one or more secondary IDH2 mutations, a therapeutically effective amount of a compound of the Formula I:

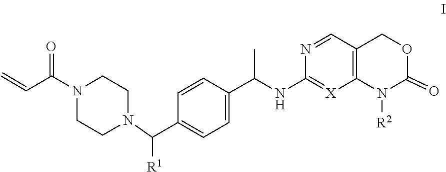

wherein:
R¹ is —CH₂CH(CH₃)₂, —CH₂CH₃, —CH₂CH₂OCH₃, or —CH₂-cyclopropyl;
R² is —CH₃ or —CH₂CH₃; and
X is N or CH;
or a pharmaceutically acceptable salt thereof.

In one embodiment of the method of the invention, in the compound of Formula I, X is N, or a pharmaceutically acceptable salt thereof. In another embodiment, X is N, R¹ is —CH₂-cyclopropyl, and R² is —CH₂CH₃, or a pharmaceutically acceptable salt thereof.

In another embodiment, X is N, R¹ is —CH₂-cyclopropyl, and R² is —CH₂CH₃.

In another embodiment, the compound of Formula I is:
7-[[(1S)-1-[4-[(1R)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one;
7-[[(1S)-1-[4-[(1S)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one; or
1-Ethyl-7-[[(1S)-1-[4-[1-(4-prop-2-enoylpiperazin-1-yl)propyl]phenyl]ethyl]amino]-4H-pyrimido[4,5-d][1,3]oxazin-2-one;
   or a pharmaceutically acceptable salt of any one thereof.

In another embodiment, the compound of Formula I is 7-[[(1S)-1-[4-[(1S)-2-cyclopropyl-1-(4-prop-2-enoylpiperazin-1-yl)ethyl]phenyl]ethyl]amino]-1-ethyl-4H-pyrimido[4,5-d][1,3]oxazin-2-one.

In another embodiment, the compound of Formula I is:

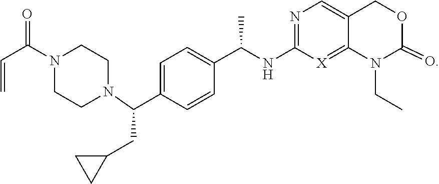

(referred to herein as “Compound A”), or a pharmaceutically acceptable salt thereof. In another embodiment, the compound is Compound A.

The present invention also provides a compound of Formula I:

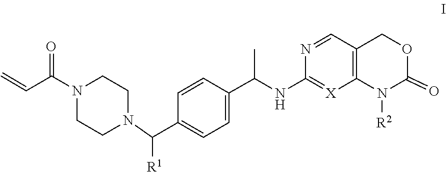

wherein:
R¹ is —CH₂CH(CH₃)₂, —CH₂CH₃, —CH₂CH₂OCH₃, or —CH₂-cyclopropyl;
R² is —CH₃ or —CH₂CH₃;
X is N or CH; or a pharmaceutically acceptable salt thereof;
for use in treating cancer in a human subject having an IDH2 R140 mutation or IDH2 R172 mutation, and one or more secondary IDH2 mutations.

For the compound of Formula I, it is preferred that X is N, or a pharmaceutically acceptable salt thereof; it is preferred that R¹ is —CH₂-cyclopropyl, or a pharmaceutically acceptable salt thereof; it is preferred that R² is —CH₂CH₃, or a pharmaceutically acceptable salt thereof; it is more preferred that X is N, R¹ is —CH₂-cyclopropyl, and R² is —CH₂CH₃, or a pharmaceutically acceptable salt thereof; it is most preferred that X is N, R¹ is —CH₂-cyclopropyl, and R² is —CH₂CH₃.

Preferred compounds of Formula I are:
7-[[(1S)-1-[4-[(1R)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one;
7-[[(1S)-1-[4-[(1S)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one; or
1-Ethyl-7-[[(1S)-1-[4-[1-(4-prop-2-enoylpiperazin-1-yl)propyl]phenyl]ethyl]amino]-4H-pyrimido[4,5-d][1,3]oxazin-2-one;
   or a pharmaceutically acceptable salt of any one thereof.

A more preferred compound of Formula I is:

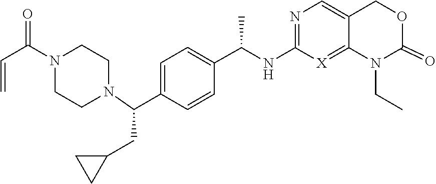

(Compound A), or a pharmaceutically acceptable salt thereof.

In one embodiment, the IDH2 mutation is an R140 mutation. In another embodiment, the R140 mutation is R140Q, R140L, or R140W. In another embodiment, the R140 mutation is R140Q. In another embodiment, the R140 mutation is R140L. In another embodiment, the R140 mutation is R140W.

In another embodiment, the IDH2 mutation is an R172 mutation. In another embodiment, the R172 mutation is R172K, R172M, R172G, R172S, or R172W. In another embodiment, the R172 mutation is R172K. In another embodiment, the R172 mutation is R172M. In another embodiment, the R172 mutation is R172G. In another embodiment, the R172 mutation is R172S. In another embodiment, the R172 mutation is R172W.

In one embodiment, the one or more secondary IDH2 mutations is Q316E, I319M, P167R, or G260A. In one embodiment, the one or more secondary IDH2 mutations is Q316E or I319M. In another embodiment, the one or more secondary mutations is Q316E. In another embodiment, the one or more secondary mutations is 1319M. In another embodiment, the one or more secondary mutations is P167R. In another embodiment, the one or more secondary mutations is G260A. In another embodiment, the one or more secondary IDH2 mutations is Q316E, I319M, and G260A. In another embodiment, the one or more secondary IDH2 mutations is Q316E and I319M.

In another embodiment, the subject is identified as having an IDH2 R140 mutation or IDH2 R172 mutation. In another embodiment, the subject is identified as having an IDH2 R140 mutation. In another embodiment, the subject is identified as having an IDH2 R172 mutation.

In another embodiment, the subject is identified as having an IDH2 R140 mutation in tissue or IDH2 R172 mutation in tissue. In another embodiment, the subject is identified as having an IDH2 R140 mutation in tissue. In another embodiment, the subject is identified as having an IDH2 R172 mutation in tissue.

In another embodiment, the subject is identified as having one or more secondary IDH2 mutations.

In another embodiment, the cancer is a hematologic malignancy, and the subject is identified as having an IDH2 R140 mutation or IDH2 R172 mutation, in blood, bone marrow, lymph node or lymphatic fluid. In another embodiment, the subject is identified as having an IDH2 R140 mutation or IDH2 R172 mutation, in blood cells, bone marrow cells, or blood cells, or lymph node cells, or lymphatic fluid cells. In another embodiment, the subject is identified as having one or more secondary IDH2 mutations.

In another embodiment, the cancer is a solid tumor cancer, and the subject is identified as having an IDH2 R140 mutation or IDH2 R172 mutation, in solid tumor tissue. In another embodiment, the solid tumor tissue is cholangiocarcinoma tissue. In another embodiment, the subject is identified as having an IDH2 R140 mutation or IDH2 R172 mutation, in solid tumor tissue cells. In another embodiment, the subject is identified as having one or more secondary IDH2 mutations.

In one embodiment, the cancer is a solid tumor. In another embodiment, the solid tumor is cholangiocarcinoma, head & neck cancer, chondrosarcoma, hepatocellular carcinoma, melanoma, pancreatic cancer, astrocytoma, oligodendroglioma, glioma, glioblastoma, bladder carcinoma, colorectal cancer, lung cancer, or sinonasal undifferentiated carcinoma. In another embodiment, the lung cancer is non-small cell lung cancer. In another embodiment, the solid tumor is cholangiocarcinoma. In another embodiment, the solid tumor is glioma.

In another embodiment, the cancer is a hematologic malignancy.

In another embodiment, the hematologic malignancy is acute myeloid leukemia, myelodysplastic syndrome myeloproliferative neoplasm, angioimmunoblastic T-cell lymphoma, T-cell acute lymphoblastic leukemia, polycythemia vera, essential thrombocythemia, primary myelofibrosis or chronic myelogenous leukemia. In another embodiment, the hematologic malignancy is acute myeloid leukemia.

In another embodiment, the subject has been treated with a mutant IDH2 inhibitor other than a compound of Formula I. In another embodiment, the mutant IDH2 inhibitor other than a compound of Formula I is enasidenib. In another embodiment, the subject has been treated with a mutant IDH2 inhibitor other than a compound of Formula I prior to treatment with a compound of Formula I.

In another embodiment, the cancer is relapsed cancer. In another embodiment, the relapsed cancer is a solid tumor cancer. In another embodiment, the relapsed solid tumor cancer is cholangiocarcinoma. In another embodiment, the relapsed cancer is hematologic malignancy. In another embodiment, the relapsed hematologic malignancy is relapsed AML.

In another embodiment, the cancer is refractory cancer. In another embodiment, the refractory cancer is a solid tumor cancer. In another embodiment, the refractory solid tumor cancer is cholangiocarcinoma. In another embodiment, the refractory cancer is hematologic malignancy. In another embodiment, the refractory hematologic malignancy is refractory AML.

In another embodiment, the cancer is advanced cancer. In another embodiment, the advanced cancer is an advanced solid tumor cancer. In another embodiment, the advanced solid tumor cancer is cholangiocarcinoma. In another embodiment, the advanced cancer is an advanced hematologic malignancy. In another embodiment, the advanced hematologic malignancy is advanced AML.

In another embodiment, the AML is acute promyelocytic leukemia.

As used above, and throughout the description of the invention, the following terms, unless otherwise indicated, shall be understood to have the following meanings:

The term “hematologic tissue” refers to blood, bone marrow, spleen, lymph node, or lymphatic fluid.

The term “solid tumor tissue” refers to tissue that is not hematologic tissue. Non-limiting examples of solid tissue are cholangial tissue, pancreatic tissue, head tissue, neck tissue, hepatic tissue, skin tissue, astrocytomal tissue, oligodendroglial tissue, glial tissue, brain tissue, bladder tissue, colorectal tissue, lung tissue, and sinonasal undifferentiated carcinoma.

The term “solid tumor cancer” means that the cancer originated in a tissue that is not blood, bone marrow, lymph node or lymphatic fluid.

The term “hematologic malignancy” relates to cancer that in the blood, the bone marrow, the lymph node or the lymphatic fluid.

The term “advanced hematological malignancy” refers to malignancy that has spread to lymph nodes or to other tissues outside of the blood or the bone marrow.

The term “cancer subject” means a subject who has been diagnosed with cancer.

The term “refractory cancer” refers to cancer that has been treated, but the human cancer subject did not respond to treatment.

The term “relapsed cancer” means that the human cancer subject responded to treatment for a period of time, but that the cancer has reoccurred.

The term “advanced cancer” refers to cancer that has spread to lymph nodes or to other tissues outside of the cancer's point of origin. For example, advanced acute myeloid leukemia is acute myeloid leukemia that has spread to a tissue outside of the blood or the bone marrow.

The term “solid tumor subject” means a subject who has been diagnosed with a solid tumor cancer. In one embodiment, the solid tumor cancer is cholangiocarcinoma.

The term “hematologic malignancy subject” means a subject who has been diagnosed with a hematologic malignancy. In one embodiment, the hematologic malignancy subject is an AML subject. The term “AML subject” means a subject who has been diagnosed with AML. Methods for diagnosing AML are known to those of ordinary skill in the art, e.g., in Dohner H, et al., Blood 2017; 129: 424-447.

The terms “acute myeloid leukemia,” “acute myelogenous leukemia,” and “acute nonlymphocytic leukemia” are synonymous.

“Responsiveness to hematologic malignancy (e.g., AML) treatment” includes improvement in overall survival, partial response, long-term stable disease, or improvement in long-term survival characterized as complete remission (determined by less than 5% myeloblasts in bone marrow, the absence of circulating blasts, hematologic recovery (as evidenced by a peripheral blood absolute neutrophil count greater than 1,000 cells/μL and a platelet count greater than 100,000/μL, without the need for red blood cell transfusion, and the absence of extramedullary disease) (Bloomfield C D, et al., Blood Revs. 2018; 32: 416-425).

The term “IDH2 R140 mutation” refers to an IDH2 mutation at amino acid residue 140 in a subject's IDH2 enzyme, as determined, e.g., in the subject's nucleic acid (e.g., DNA). As used herein, an “IDH2 R140 mutation” is not a “secondary IDH2 mutation.”

The term “IDH2 R172 mutation” refers to an IDH2 mutation at amino acid residue 172 in a subject's IDH2 enzyme, as determined, e.g., in the subject's nucleic acid (e.g., DNA). As used herein, an “IDH2 R172 mutation” is not a “secondary IDH2 mutation.”

The term “secondary IDH2 mutation” refers to an IDH2 mutation that occurs in the IDH2 enzyme in a human subject after treatment with a mutant IDH2 inhibitor other than a compound of Formula I herein. In one embodiment, the one or more secondary IDH2 mutations is one or more of Q316E, I319M, P167R, or G260A in IDH2. In one embodiment, the one or more secondary IDH2 mutations is one or more of Q316E or I319M in IDH2. However, other secondary IDH2 mutations may be reported in the future. As used herein, a “secondary IDH2 mutation” is not an “IDH2 R140 mutation” or “IDH2 R172 mutation.”

The term “mutant IDH2 inhibitor” refers to a compound that inhibits the enzyme activity of and/or the production of 2-HG by a mutant IDH2 enzyme. Methods for assaying mutant IDH2 enzyme activity are known to those of ordinary skill in the art, e.g., in WO 2018/111707 A1. In the term “mutant IDH2 inhibitor,” the word “mutant” refers to the IDH2 gene, not the inhibitor.

The term “identified as having an IDH2 R140 mutation” means that nucleic acid (e.g., DNA) from a human subject's tissue or cells has been analyzed to determine if the human subject has an IDH2 R140 mutation. In one embodiment, one or more of the human subject's blood cells, bone marrow cells, lymph node, lymph node cells, lymphatic fluid or lymphatic fluid cells has been analyzed for an IDH2 R140 mutation. In another embodiment, the human subject's solid tissue has been analyzed for an IDH2 R140 mutation.

The term “identified as having an IDH2 R172 mutation” means that nucleic acid (e.g., DNA) from a human subject's tissue or cells has been analyzed to determine if the human subject has an IDH2 R172 mutation. In one embodiment, one or more of the human subject's blood cells, bone marrow cells, lymph node, lymph node cells, lymphatic fluid or lymphatic fluid cells has been analyzed for an IDH2 R172 mutation. In another embodiment, the human subject's solid tissue has been analyzed for an IDH2 R172 mutation.

In the method of the present invention, the party who identifies the human subject as having an IDH2 R140 mutation or IDH2 R172 mutation can be different than the party that administers the compound. In one embodiment, the party who identifies the human subject as having an IDH2 R140 mutation or IDH2 R172 mutation is different than the party that administers the compound.

The term “identified as having one or more secondary IDH2 mutation(s)” means that nucleic acid (e.g., DNA) from one or more of the human subject's blood cells, bone marrow cells, lymph node, lymph node cells, lymphatic fluid or lymphatic fluid cells has been analyzed to determine if a human subject has one or more secondary IDH2 mutation(s).

Analytical methods for identifying genetic mutations are known to those of ordinary skill in the art (Clark, O., et al., Clin. Cancer. Res. 2016; 22: 1837-42), including, but not limited to, karyotyping (Guller J L, et al., J. Mol. Diagn. 2010; 12: 3-16), fluorescence in situ hybridization (Yeung D T, et al., Pathology 2011; 43: 566-579), Sanger sequencing (Lutha, R et al., Haematologica 2014; 99: 465-473), metabolic profiling (Miyata S, et al., Scientific Reports 2019; 9: 9787), polymerase chain reaction (Ziai, J M and AJ Siddon, Am. J. Clin. Pathol 2015; 144: 539-554), and next-generation sequencing (e.g., whole transcriptome sequencing) (Lutha, R et al., Haematologica 2014; 99: 465-473; Wang H-Y, et al., J. Exp. Clin. Cancer Res. 2016; 35: 86).

The terms “treatment,” “treat,” “treating,” and the like, are meant to include slowing, stopping, or reversing the progression of cancer. These terms also include alleviating, ameliorating, attenuating, eliminating, or reducing one or more symptoms of a disorder or condition, even if the cancer is not actually eliminated and even if progression of the cancer is not itself slowed, stopped or reversed.

“Therapeutically effective amount” means the amount of a compound, or pharmaceutically acceptable salt thereof, administered to the subject that will elicit the biological or medical response of or desired therapeutic effect on a subject. A therapeutically effective amount can be readily determined by the attending clinician, as one skilled in the art, by the use of known techniques and by observing results obtained under analogous circumstances. In determining the effective amount for a subject, a number of factors are considered by the attending clinician, including, but not limited to: size, age, and general health of the subject; the specific disease or disorder involved; the degree of or involvement or the severity of the disease or disorder; the response of the individual subject; the particular compound administered; the mode of administration; the bioavailability characteristics of the preparation administered; the dose regimen selected; the use of concomitant medication; and other relevant circumstances.

A compound of Formula I herein can optionally be formulated as a pharmaceutical composition administered by any route which makes the compound bioavailable, including oral, intravenous, and transdermal routes. It is preferred that such compositions are formulated for oral administration. Such pharmaceutical compositions and processes for preparing the same are well known in the art. (See, e.g., Remington: The Science and Practice of Pharmacy (D. B. Troy, Editor, 21st Edition, Lippincott, Williams & Wilkins, 2006).

A “pharmaceutically acceptable carrier, diluent, or excipient” is a medium generally accepted in the art for the delivery of biologically active agents to mammals, e.g., humans.

It will be understood by one of ordinary skill in the art that compounds administered in the method of the invention are capable of forming salts. The compounds react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008).

“Pharmaceutically acceptable salts” or “a pharmaceutically acceptable salt” refers to the relatively non-toxic, inorganic and organic salt or salts of the compounds of the present invention (S. M. Berge, et al., “Pharmaceutical Salts”, Journal of Pharmaceutical Sciences, Vol 66, No. 1, January 1977).

### MATERIALS, METHODS AND RESULTS

Compounds and Formulation. Compound A may be synthesized as described in WO/18/111707. Compound A is prepared as a 20 mM stock in 100% DMSO (dimethyl sulfoxide) (Sigma, D2438) and diluted serially in 100% DMSO to achieve the desired concentrations. DMSO preparations are further diluted with cell culture media prior to addition in the assay.

Cell lines. U-87 MG cells (ATCC, HTB-14) are cultured and assayed in MEM (Gibco, 11095) with 2 mM GlutaMAX (Gibco, 35050), 1 mM Pyruvate (Gibco, 11360), 0.1 mM NEAA ((Non-essential amino acid) Gibco, 11140) and 10% dialyzed FBS (Fetal bovine serum) (Gibco, 26400).

Cell-based inhibition assays. Cell-based assays are performed by measuring 2-HG in U-87 MG cells in which IDH2 mutations are expressed.

DNA constructs encoding IDH2 mutations are introduced into U-87 MG cells using transfection (Promega FuGENE HD, E2311) or lentiviral transduction, and the IDH2-mutant expressing cell lines are selected using blasticidin (5 μg/ml) or puromycin (1 μg/ml). Second site mutations (Q316E, I319M, P167R, and G260A) are evaluated in cis (same monomer as driver mutation within dimer) or in trans (opposite monomer from driver mutation in dimer). For compound treatments, 12,500 or 50,000 cells per well in culture medium (150 μl or 100 μl, respectively) are plated in 96 well cell culture plates (Falcon, 353377) 2 hrs prior to treatment. Cells are treated with serial dilutions of compound A in standard growth media. Plates are incubated in a mammalian cell culture incubator (humidified, 37° C., 5% CO₂) for 16 or 72 hrs. Following the incubation period, the media is aspirated and cell extracts are prepared by addition of ice-cold 80% methanol/20% water (100 μL) containing liquid chromatography-mass spectrometry (LC-MS) internal standards (1 μM ¹³C₄ α-KG and ¹³C₅ 2-HG) per well for ion pairing LC-MS method. 96-well sample plates are then sealed, shaken at 450 rpm for 10 min, and then placed at −20° C. and stored until LC-MS analysis.

LC-MS metabolite analysis of conditioned media and cell lysates. The effect of inhibitors on the concentrations of 2-HG are determined by LC-MS analysis of cell lysates using an ion-pairing method as described below.

Calibration curves are prepared by spiking 2-HG and α-KG into 80% methanol/20% water containing LC-MS internal standards (1 μM ¹³C₄ α-KG and 1 μM ¹³C₅ 2-HG). The quantitation of 2-HG and α-KG is accomplished using an AB Sciex 6500 mass spectrometer with an Electrospray Ionization (ESI) probe and interfaced with an Ultra Performance Liquid Chromatography (UHPLC) system in the negative multiple-reaction monitoring (MRM) mode. The UHPLC system consists of an Agilent 1290 binary pump, thermostatted column compartment (TCC), and sampler. The injection volume is 1 μL for cell culture extracts. The extracts are chromatographically resolved using a Hypercarb column, 2.1×20 mm, 5.0 mm Javelin HTS (Thermo Scientific, PN: 35005-022135). Mobile phase A is water/10 mM tributylamine/15 mM acetic acid. Mobile phase B is acetonitrile/20 mM tributylamine/30 mM acetic acid. The solvent flow rate is 1.0 mL/min. The isocratic condition is kept at 26% mobile phase B. The valve, sample loop, and needle are washed with 50% acetonitrile: 50% methanol for 20 seconds. The column temperature is kept at 55° C. Calibration curves are calculated by least-square linear regression with 1/x weighting. 2-HG and α-KG are quantified using standard curve and ratio of the peak area of analytes to internal standard. Data analysis is performed using MultiQuant 3.0 (AB Sciex). The raw data are exported to Microsoft Excel spreadsheets.

Determination of IC₅₀ Curves. IC₅₀ curves for each compound are obtained using four parameter data fitting analysis in GraphPad/Prism software.

In experiments performed essentially as described above, the IC₅₀ results set forth in Tables 1-5 were obtained.

TABLE 1
Inhibition of 2-HG Production in IDH2 Mutant Cells
by Compound A at 72 Hours Incubation Time

| | | Compound A | Std | |
|---|---|---|---|---|
| Cell-line | Mutation | IC₅₀ (nM) | Dev | n |
| U-87 MG | IDH2 R140Q | 28.39 | 1.47 | 8 |
| U-87 MG | IDH2 R172K | 13.52 | 0.73 | 8 |
| U-87 MG | IDH2 R140Q_Q316E cis | 21.96 | 1.07 | 8 |
| U-87 MG | IDH2 R140Q + Q316E trans | 5.84 | 0.38 | 8 |
| U-87 MG | IDH2 R140Q_I319M cis | 30.37 | 2.0 | 8 |
| U-87 MG | IDH2 R140Q + 1319M trans | 12.72 | 0.75 | 8 |

Std Dev refers to standard deviation, n refers to number of assay runs.
12.5K cells/well; 150 μl media/well

The results in Table 1 indicate that compound A is active against IDH2 driver mutations (R140Q and R172K) and IDH2 second-site mutations, Q316E and I319M, associated with resistance to mutant IDH2 inhibitor enasidenib.

TABLE 2
Inhibition of 2-HG Production in IDH2 R140Q Mutant
Cells by Compound A at 16 Hours Incubation Time

| | | Compound A | | |
|---|---|---|---|---|
| | | Relative IC₅₀ | Std | |
| Cell-line | Mutation | (nM) Mean | Dev | n |
| U-87 MG | IDH2 R140Q | 223 | 40 | 6 |
| U-87 MG | IDH2 R140Q_I319M cis | 29 | 1 | 2 |
| U-87 MG | IDH2 R140Q_I319M trans | 18* | — | 1 |
| U-87 MG | IDH2 R140Q_Q316E cis | 16 | — | 1 |
| U-87 MG | IDH2 R140Q_Q316E trans | 23 | — | 1 |
| U-87 MG | IDH2 R140Q_P167R cis | 639 | 23 | 2 |
| U-87 MG | IDH2 R140Q_P167R cis | 104* | 32 | 2 |
| U-87 MG | IDH2 R140Q_G260A cis | 7.0 | 0.6 | 4 |

Std Dev refers to standard deviation, n refers to number of assay runs.
50K cells/well; 100 μl media/well
*IC₅₀ at 72 hours incubation time; 12.5K cells/well

The results in Table 2 indicate that compound A is active against IDH2 driver mutation R140Q and IDH2 second-site mutations I319M, Q316E, P167R, and G260A.

TABLE 3
Inhibition of 2-HG Production in IDH2 R140L Mutant
Cells by Compound A at 16 Hours Incubation Time

| | | Compound A | | |
|---|---|---|---|---|
| | | Relative IC₅₀ | Std | |
| Cell-line | Mutation | (nM) Mean | Dev | n |
| U-87 MG | IDH2 R140L | 243 | 9 | 4 |
| U-87 MG | IDH2 R140L_Q316E cis | 145 | 24 | 4 |
| U-87 MG | IDH2 R140L_Q316E trans | 262 | 13 | 4 |

Std Dev refers to standard deviation, n refers to number of assay runs.
50K cells/well; 100 μl media/well

The results in Table 3 indicate that compound A is active against IDH2 driver mutation R140L and IDH2 second-site mutation Q316E.

TABLE 4
Inhibition of 2-HG Production in IDH2 R172K Mutant
Cells by Compound A at 16 Hours Incubation Time

| | | Compound A | | |
|---|---|---|---|---|
| | | Relative IC₅₀ | Std | |
| Cell-line | Mutation | (nM) Mean | Dev | n |
| U-87 MG | IDH2 R172K | 56 | 7 | 6 |
| U-87 MG | IDH2 R172K_Q316E cis | 76 | 19 | 2 |
| U-87 MG | IDH2 R172K_P167R cis | 119 | 15 | 2 |
| U-87 MG | IDH2 R172K_G260A cis | 13 | 3 | 4 |

Std Dev refers to standard deviation, n refers to number of assay runs.
50K cells/well; 100 μl media/well

The results in Table 4 indicate that compound A is active against IDH2 driver mutation R172K and IDH2 second-site mutations Q316E, P167R, and G260A.

TABLE 5
Inhibition of 2-HG Production in IDH2 R172W Mutant
Cells by Compound A at 16 Hours Incubation Time

| | | Compound A | | |
|---|---|---|---|---|
| | | Relative IC₅₀ | Std | |
| Cell-line | Mutation | (nM) Mean | Dev | n |
| U-87 MG | IDH2 R172W | 336 | 5 | 2 |
| U-87 MG | IDH2 R172W | 47* | 5 | 2 |
| U-87 MG | IDH2 R172W_P167R cis | 1280 | 64 | 2 |
| U-87 MG | IDH2 R172W_P167R cis | 535* | 58 | 2 |

Std Dev refers to standard deviation, n refers to number of assay runs.
50K cells/well; 100 μl media/well
*IC₅₀ at 72 hours incubation time; 12.5K cells/well

The results in Table 5 indicate that compound A is active against IDH2 driver mutation R172W and IDH2 second-site mutation P167R.
Compound A is currently in phase 1 studies for advanced hematologic malignancies (NCT04603001) and advanced solid tumors (NCT04521686). Preliminary data shows Compound A administration leads to 2-HG inhibition in 2 AML patients who received prior enasidenib treatment and have an IDH2 second site mutation: one patient with an IDH2 R172K mutation plus a G260A second site mutation; the other patient with an IDH1 mutation, and an IDH2 R140L mutation plus a Q316E second site mutation.

1. A method of treating cancer, comprising administering to a human cancer subject having an IDH2 R140 mutation or IDH2 R172 mutation, and one or more secondary IDH2 mutations, a therapeutically effective amount of a compound of the formula: 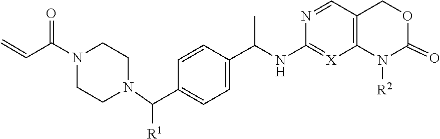
wherein:
R¹ is —CH₂CH(CH₃)₂, —CH₂CH₃, —CH₂CH₂OCH₃, or —CH₂-cyclopropyl;
R² is —CH₃ or —CH₂CH₃; and
X is N or CH;
or a pharmaceutically acceptable salt thereof.

2. The method of claim 1, wherein X is N, or a pharmaceutically acceptable salt thereof.

3. The method of claim 1, wherein X is N, R¹ is —CH₂-cyclopropyl, and R² is —CH₂CH₃, or a pharmaceutically acceptable salt thereof.

4. The method of claim 1, wherein the compound is:
7-[[(1S)-1-[4-[(1R)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one;
7-[[(1S)-1-[4-[(1S)-2-Cyclopropyl-1-(4-prop-2-enoylpiperazin-1-yl)ethyl]phenyl]ethyl]amino]-1-ethyl-4H-pyrimido[4,5-d][1,3]oxazin-2-one; or
1-Ethyl-7-[[(1S)-1-[4-[1-(4-prop-2-enoylpiperazin-1-yl)propyl]phenyl]ethyl]amino]-4H-pyrimido[4,5-d][1,3]oxazin-2-one;
or a pharmaceutically acceptable salt thereof.

5. The method of claim 1, wherein the compound is: 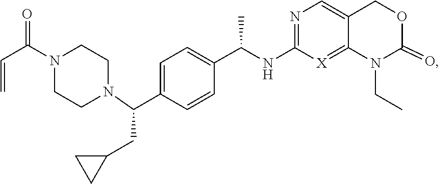
or a pharmaceutically acceptable salt thereof.

6. The method of claim 5, wherein the compound is:

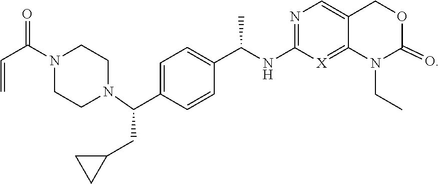

7. The method of claim 1, wherein the cancer is a solid tumor cancer.

8. The method of claim 7, wherein the solid tumor cancer is cholangiocarcinoma, head and neck cancer, chondrosarcoma, hepatocellular carcinoma, melanoma, pancreatic cancer, astrocytoma, oligodendroglioma, glioma, glioblastoma, bladder carcinoma, colorectal cancer, lung cancer, or sinonasal undifferentiated carcinoma.

9. The method of claim 8, wherein the solid tumor cancer is cholangiocarcinoma.

10. The method of claim 1, wherein the compound is 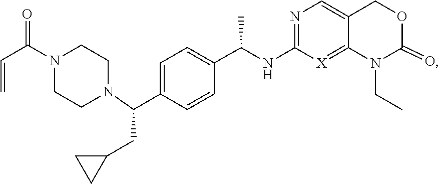
and the cancer is cholangiocarcinoma.

11. The method of claim 1, wherein the cancer is a hematologic malignancy.

12. The method of claim 11, wherein the hematologic malignancy is acute myeloid leukemia, myelodysplastic syndrome myeloproliferative neoplasm, angioimmunoblastic T-cell lymphoma, T-cell acute lymphoblastic leukemia, polycythemia vera, essential thrombocythemia, primary myelofibrosis, or chronic myelogenous leukemia.

13. The method of claim 12, wherein the hematologic malignancy is acute myeloid leukemia.

14. The method of claim 1, wherein the compound is 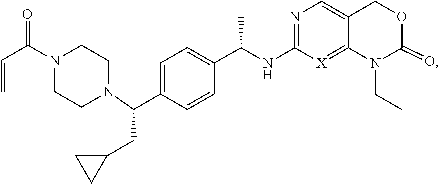
and the cancer is acute myeloid leukemia.

15. The method of claim 1, wherein the human cancer subject has an IDH2 R140 mutation.

16. The method of claim 1, wherein the human cancer subject has an IDH2 R172 mutation.

17. The method of claim 1, wherein the one or more secondary IDH2 mutations is one or more of Q316E, I319M, P167R, or G260A.

18. The method of claim 1, wherein the one or more secondary IDH2 mutations is one or more of Q316E or I319M.

19. The method of claim 1, wherein the human subject has been treated with an IDH2 inhibitor other than a compound of the formula: 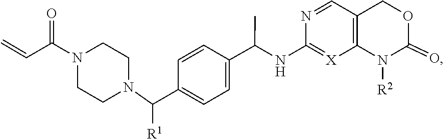
wherein:
R¹ is —CH₂CH(CH₃)₂, —CH₂CH₃, —CH₂CH₂OCH₃, or —CH₂-cyclopropyl;
R² is —CH₃ or —CH₂CH₃; and
X is N or CH;
or a pharmaceutically acceptable salt thereof.

20. The method of claim 19, wherein the human subject has been treated with enasidenib.

21.-54. (canceled)
